SEQUENCE LISTING (1 sequence)

SEQ ID NO: 1 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

(19) United States
(12) Patent Application Publication
(10) Pub. No.: US 2013/0217595 A1
(43) Pub. Date: Aug. 22, 2013

(54) METHOD FOR THE IDENTIFICATION OF CATECHOL O-METHYLTRANSFERASE MODULATORS

(71) Applicant: Hoffman-La Roche Inc., (US)

(72) Inventors: Thilo Enderle, Rheinfelden (DE); Doris Roth, Allschwil (CH)

(73) Assignee: Hoffman-La Roche Inc., Nutley, NJ (US)

(21) Appl. No.: 13/750,967

(22) Filed: Jan. 25, 2013

Related U.S. Application Data
(63) Continuation of application No. PCT/EP2011/062704, filed on Jul. 25, 2011.

(30) Foreign Application Priority Data
Jul. 27, 2010 (EP) 10170957.4

(51) Int. Cl. C12Q 1/48

(52) U.S. Cl. CPC C12Q 1/48
USPC 506/11; 435/15

(57) ABSTRACT

The present invention relates to a method for the identification of modulators of catechol O-methyltransferase enzyme activity (COMT).

### CROSS REFERENCE TO RELATED APPLICATIONS

This application is a continuation of International Application No. PCT/EP2011/062704 having an international filing date of Jul. 25, 2011, the entire contents of which are incorporated herein by reference and which claims benefit under 35 U.S.C. §119 to European Patent Application No. 10170957.4 filed Jul. 27, 2010.

### SEQUENCE LISTING

The instant application contains a Sequence Listing submitted via EFS-Web and is hereby incorporated by reference in its entirety. Said ASCII copy, created on Jan. 25, 2013, is named P4677C1SeqList.txt, and is 3 bytes in size.

### FIELD OF THE INVENTION

The present invention relates to a method for the identification of modulators of catechol O-methyltransferase enzyme activity.

### BACKGROUND

Catechol O-methyltransferase (COMT) catalyzes the O-methlyation of substrates that have a catechol moiety. The methyl donor in the methylation by COMT is S-adenosylmethionine (SAM). COMT plays an important role for the catabolism of endogenous catecholamine neurotransmitters, catecholestrogens and xenobiotic molecules. Inhibition of COMT is an important approach for developing new therapeutic treatments in Parkinson's disease.

W. F. Herblin (Analytical Biochemistry 51, 19-22, 1973) describes a colorimetric assay for COMT activity. The assay uses nitrocatechol as methyl acceptor for COMT. Nitrocatechol exists as a yellow solution in water at acid pH with an absorption maximum at 350 nm. At slightly alkaline pH ionization of the para hydroxyl turns the solution orange (λmax=430 nm). In stronger alkali ionization of the meta hydroxyl leads to a cherry red solution (λmax=520 nm). The assay is based on the observations that nitrocatechol is methylated by COMT and that methylated nitrocatechol no longer exhibits the cherry red color resulting from the second ionization. In this assay the substrate (nitrocatechol) and SAM have to be in the μM concentration range which is at or above Kₘ what is limiting the sensitivity.

G. Zürcher and M. Da Prada (Journal of Neurochemistry, Vol. 38, No. 1, 1982) describe a Single step radiochemical assay for COMT activity. In this assay, catechol is converted to tritiated guajacol, a compound of very low polarity, by incubating COMT with [³H]methyl SAM, Mg²⁺ and adenosine deaminase. Guajacol is extracted using a low polarity medium e.g. Toluene, and counted in a scintillation counter.

The above described assays are not suitable for automated screening large numbers of compounds for their COMT modulator activity due to limited sensitivity (colorimetric assay) or due to the assay setup (extraction step in the radiochemical assay).

Therefore, there is a need for a sensitive, homogeneous assay method suitable for screening large numbers of compounds for their COMT modulating activity.

### SUMMARY OF THE INVENTION

In a first object the present invention provides a method for the identification of a modulator of the activity of a catechol O-methyl transferase enzyme (COMT) comprising the steps of:
a) providing a COMT substrate covalently linked to a fluorescence dye,
b) contacting the molecule of step a) with a catechol O-methyl transferase enzyme (COMT), a S-adenosylmethionine (SAM) and a candidate compound and
c) measuring the fluorescence readout of the mixture of step b), wherein an altered fluorescence readout in presence of the candidate compound compared to a blank is indicative for a modulator of a catechol O-methyl transferase enzyme (COMT).

In a preferred embodiment, the method is a method for the identification of a COMT inhibitor, wherein a decreased fluorescence readout in step c) compared to a blank is indicative for a COMT inhibitor.

In a further preferred embodiment the COMT substrate is 4-nitrocatechol.

In a further preferred embodiment the fluorescence dye is Alexa Fluor® 488

In a further preferred embodiment the fluorescence readout in step c) is a kinetic readout.

In a further preferred embodiment the COMT is human COMT.

In a further preferred embodiment the method is a High Throughput screening method.

In a further preferred embodiment the method is performed in a microtiter plate.

In a further preferred embodiment the final concentration of COMT is about 25 nM.

In a further preferred embodiment the final concentration of the COMT substrate is about 200 nM.

In a further preferred embodiment the final concentration of SAM is about 500 nM.

In a second object, the present invention provides a method for the identification of a substrate of a catechol O-methyl transferase enzyme (COMT) comprising the steps of:
a) providing a mixture comprising a COMT substrate covalently linked to a fluorescence dye and S-adenosylmethionine (SAM),
b) contacting the mixture of step a) with different concentrations of a candidate compound,
c) contacting the mixtures of step b) with a catechol O-methyl transferase enzyme (COMT) and
d) measuring the kinetic fluorescence readout of the mixtures of step c), wherein a decreasing fluorescence readout plateau as a function of an increasing concentration of the candidate compound is indicative for a substrate of a catechol O-methyl transferase enzyme (COMT).

In a preferred embodiment the COMT substrate is 4-nitrocatechol.

In a further preferred embodiment the fluorescence dye is Alexa Fluor® 488.

### SHORT DESCRIPTION OF THE FIGURES

FIG. 1 shows the chemical structure of Alexa Fluor® 488 covalently coupled to 4-Nitrocatechol;

FIG. 2 shows a Stern Volmer Plot for nitrocatechol (top structure), 2-methoxy-5-nitrophenol (bottom left structure) and 1,2-dimethoxy-4-nitrobenzene (bottom right structure); 20 nM free Alexa Fluor 488 was mixed with high—up to 25 mM—concentrations of nitrocatechol, 2-methoxy-5-nitrophenol and 1,2-dimethoxy-4-nitrobenzene, respectively. Only for nitrocatechol a change of the fluorescence intensity (I₀/I) of Alexa Fluor® 488 is observed, the methylated products do not influence the fluorescence intensity of Alexa Fluor® 488.

FIG. 3 shows the enzyme kinetics of the COMT catalyzed methylation of Alexa Fluor 488-Nitrocatechol in the fluorescence assay of the present invention;

FIG. 4a shows a kinetic measurement of the change of the fluorescence intensity in presence of various concentrations of the COMT inhibitor Tolcapone;

FIG. 4b shows the dose response curve for Tolcapone calculated from the slopes of the kinetic measurement of FIG. 3a;

FIG. 5 shows the fluorescent assay in presence of dopamine, a natural substrate of COMT. Decreasing plateaus were reached with increasing dopamine concentrations. As dopamine is a substrate it is methylated as well as the Alexa Fluor® 488-Nitrocatechol substrate. The availability of SAM is limited (500 nM) so that at high dopamine concentrations the Alexa Fluor® 488-Nitrocatechol substrate can no longer get fully methylated.

FIG. 6 shows the dose response curves for a substrate with low (500 nM) and high (200 μM) SAM concentrations and for each SAM concentration with and without one hour pre-incubation of compound and SAM before adding the Alexa Fluor® 488-Nitrocatechol substrate. The pre-incubation with low SAM shifts the dose response curve to a lower IC₅₀ because the compound uses up SAM. At high SAM concentrations there is no difference between with and without pre-incubation because SAM is not limiting and the dose response curve is shifted to higher IC₅₀ compared to low SAM because the compound is used up.

FIG. 7 shows dose response curves with low (500 nM) and high (200 μm) SAM concentrations again with and without one hour pre-incubation of the compound with SAM of a SAM competitive compound. For a SAM competitive compound with and without pre-incubation does not affect the IC₅₀ for each SAM concentration but with high SAM concentration the IC₅₀ is shifted to larger values.

### DETAILED DESCRIPTION OF THE INVENTION

The assay of the present invention is based on the findings that a fluorescent dye covalently coupled to a COMT substrate e.g. Alexa Fluor® 488 covalently linked to nitrocatechol, shows a decreased fluorescence due to intramolecular quenching and that methylation of the COMT substrate in the COMT substrate-fluorescent dye complex by COMT abolishes quenching of the fluorescence i.e. methylation of the COMT substrate in the COMT substrate—fluorescent dye complex leads to an increased fluorescence compared to the non methylated complex.

The term “COMT” is used herein to refer to native sequence COMT from any animal, e.g. mammalian, species, including humans, and COMT variants (which are further defined below). The COMT polypeptides may be isolated from a variety of sources, including human tissue types or prepared by recombinant and/or synthetic methods.

Natural or recombinantly produced COMT can be used in this assay. A “recombinant protein” is a protein isolated, purified, or identified by virtue of expression in a heterologous cell, said cell having been transduced or transfected, either transiently or stably, with a recombinant expression vector engineered to drive expression of the protein in the host cell. Recombinant COMT can be produced in procaryotic cells e.g. E. coli, in yeast e.g. S. pombe or in eukaryotic cells e.g. HEK 293, Sf9 insect cells. Preferably, Sf9 insect cells are used for high expression of recombinant COMT. The COMT used in the assay may be purified. The term “purified” as used herein refers to polypeptides, that are removed from their natural environment or from the source of recombinant production, isolated or separated, and are at least 60% and more preferably at least 80% free from other components, e.g. membranes and microsomes, with which they are naturally associated.

“Native sequence COMT” refers to a polypeptide having the same amino acid sequence as a COMT polypeptide occurring in nature regardless of its mode of preparation. A native sequence COMT may be isolated from nature, or prepared by recombinant and/or synthetic methods. The term “native sequence COMT” specifically encompasses naturally occurring truncated or secreted forms, naturally occurring variant forms (e.g. alternatively spliced forms), and naturally occurring allelic variants of COMT. The identifier of the human COMT polypeptide in the NCBI database is AAA68927 (Seq. Id. No. 1).

The term “COMT variant” refers to amino acid sequence variants of a native sequence COMT, containing one or more amino acid substitution and/or deletion and/or insertion in the native sequence. The amino acid sequence variants generally have at least about 75%, preferably at least about 80%, more preferably at least about 85%, even more preferably at least about 90%, most preferably at least about 95% sequence identity with the amino acid sequence of a native sequence COMT.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “kinetic readout” refers to the difference of the fluorescent signal measured at two certain time points in the linear part of an enzymatic reaction. One measurement is done at the beginning of the enzymatic reaction (start-point) and after an incubation time a second readout is performed (end-point). The final signal is then calculated in rfu/min as (rfu(end-point)—rfu(start-point))/incubation time. (rfu: relative fluorescence units)

The method of the present invention can by employed to identify compounds which inhibit the enzyme Catechol O-methyltransferase (COMT). Thus, COMT inhibitors identified by the method of the present invention can be used in methods for the treatment, prevention, or control of illnesses in which a deactivation of extraneuronal catecholamines by COMT plays a role, for example, in the prevention or control of depressions. In this case the compounds of the invention can be used as individual compounds or in combination with other therapeutically active substances which favorably influence the course of the illness. The compounds of the invention can also be used as co-medications with other therapeutically active substances.

The method of the present invention can be used to determine COMT activity in tissue samples of animals which have been treated with a test compound. For example, the assay is suitable to determine COMT activity in brain and liver tissue samples from animals e.g. mice and rats, which have been treated with a test compound (COMT modulator).

Experimental Part

Synthesis of 4-Nitrocatechol—Alexa Fluor® 488

A 10 mM solution of Aminoethyl-nitro-brenzcatechin [1] in DMSO containing 1% Triethylamine was mixed with a 10 mM solution of Alexa Fluor® 488 carboxylic acid succinimidyl ester [2] (Invitrogen Corporation, 5791 Van Allen Way, Carlsbad, Calif. 92008) in DMSO with 1% Trietylamine in a 1:1 soichiometric ratio. The reaction mixture was gently mixed over night at room temperature and purified on a Äkta Explorer 100 Reversed-Phase-HPLC. The product was lyophilized and resuspended in DMSO.

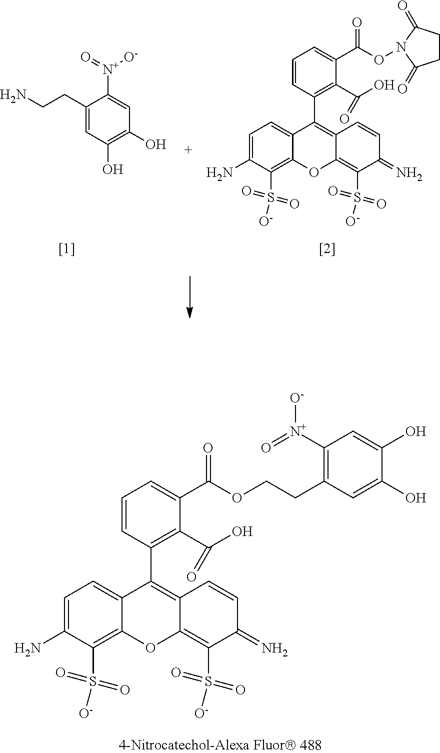 [SMILES: NCCc1cc(O)c(O)cc1[N+](=O)[O-] ; N=c1ccc2c(-c3cccc(C(=O)ON4C(=O)CCC4=O)c3C(=O)O)c3ccc(N)c(S(=O)(=O)[O-])c3oc-2c1S(=O)(=O)[O-] ; N=c1ccc2c(-c3cccc(C(=O)OCCc4cc(O)c(O)cc4[N+](=O)[O-])c3C(=O)O)c3ccc(N)c(S(=O)(=O)[O-])c3oc-2c1S(=O)(=O)[O-]]

Fluorescence Assay Protocol

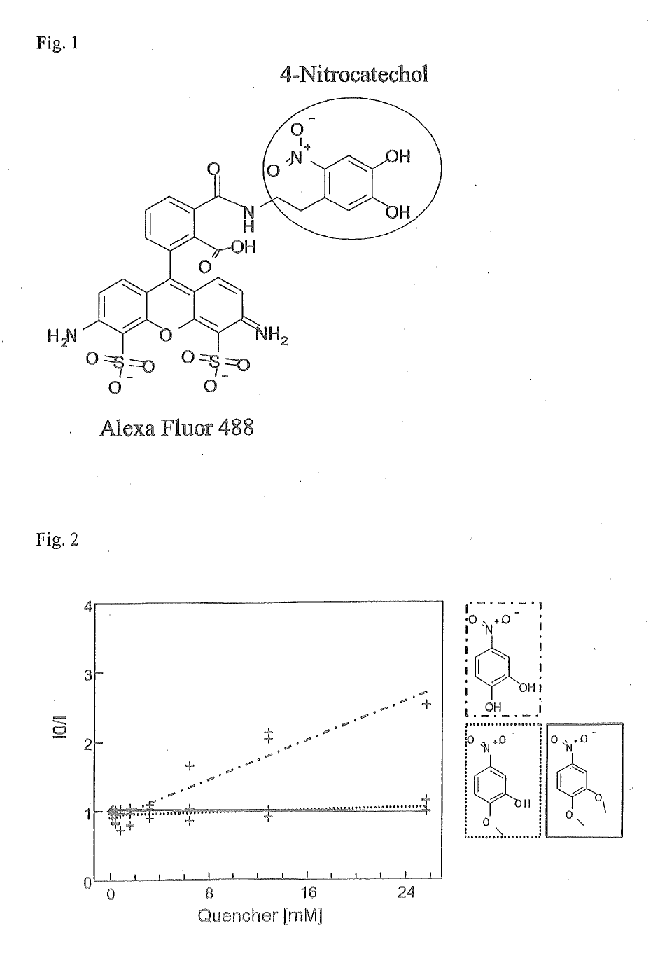

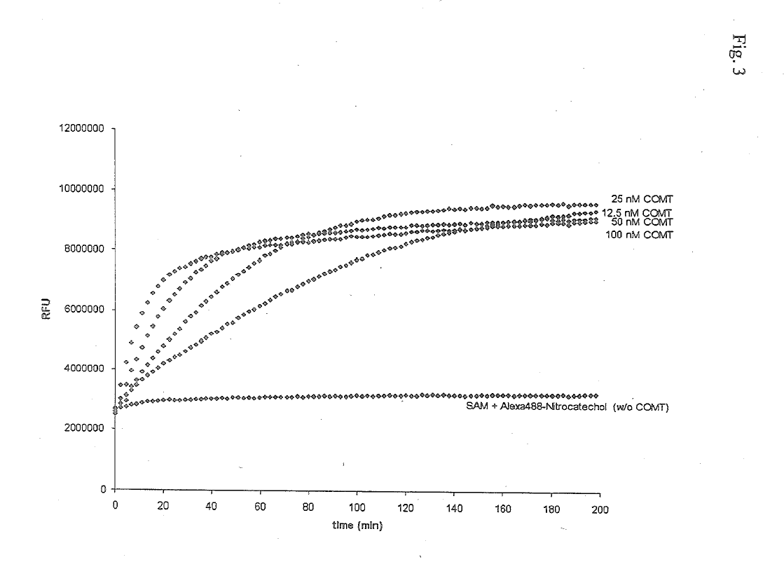

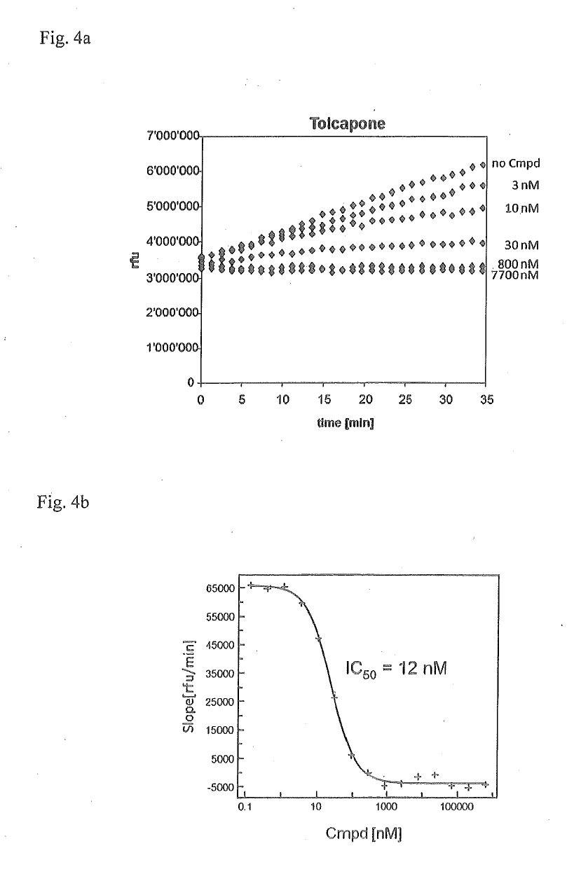

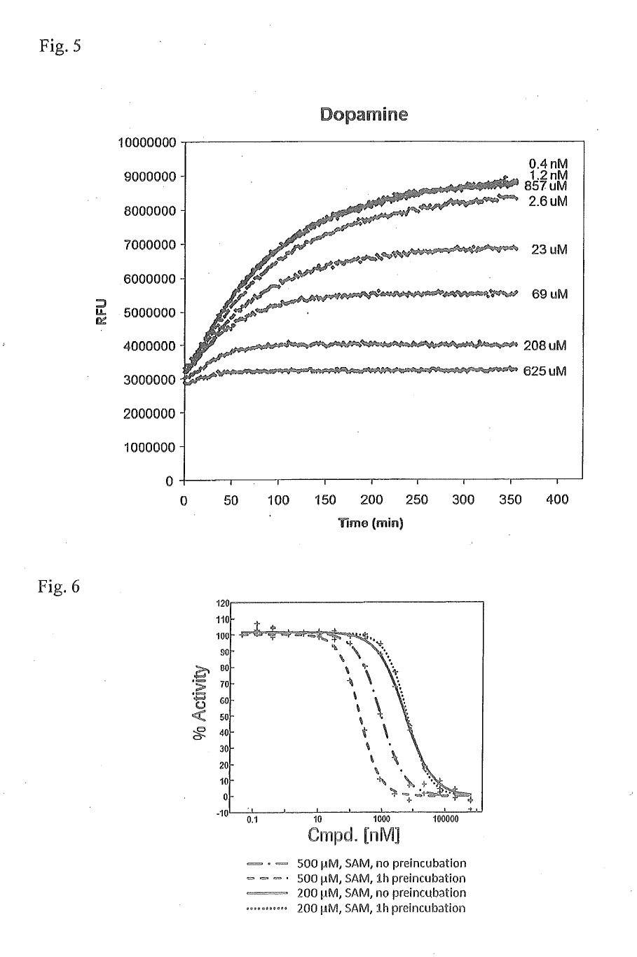

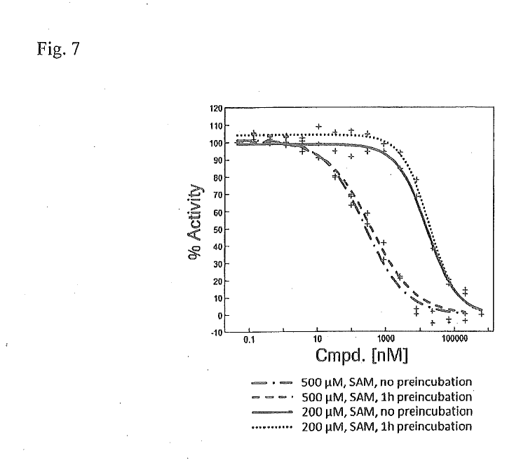

The following assay protocol, reagents and materials were used in the examples of the present invention. The results of the examples are described in the FIGS. 1-7.

Microtiterplates:

384-well microtiter plate, Corning black with flat clear bottom, non binding surface, polystyrene (ref. 3655)

Reagent and Buffer Stock Solutions:
Buffer stock solutions:
   M Phosphate buffer pH 7.6 (Na₂HPO₄ Fluka 71644, NaH₂PO₄ Merck 6346.0500) stored at 4° C.
   580 mM MgCl₂ (Merck 1.0833.0250), stored at RT
   1 M CaCl₂ stored at 4° C.
65 mM DTT (Sigma D-0632), stored at −20° C.
Recombinant human COMT: prepared in house, stored at −80° C.
4-Nitrocatechol-Alexa Fluor 488: prepared in house, 1.3 mM in DMSO, stored at RT in the dark
S-Adenosyl-methionine: 10 mM in H₂O (Sigma-Aldirch A2804), stored at −20° C.

Reagent and Buffer Solutions:
Assay buffer (end concentrations):
   40 mM Phosphate buffer pH 7.6
   2.88 mM MgCl₂
   0.9 mM DTT
   0.25 mM CaCl₂
Compound dilutions: dilutions in 100% DMSO (Sigma 41640), 6.25% DMSO final assay concentration
Rec. human COMT: 80 nM in assay buffer, 25 nM final assay concentration
4-Nitrocatechol-Alexa Fluor® 488: 320 nM in assay buffer, 200 nM final assay concentration
S-Adenosyl-methionine: 800 nM in assay buffer, 500 nM final assay concentration

Assay Method:

10 μl hCOMT (human COMT)

2 μl test compound

1 min on shaker

20 μl Substrate-SAM-Mix

5 min on shaker

Readout: kinetic measurement on plate: vision TM reader (exc. 475(40) nm, em. 535(45) nm, intensity 7.5%, exposure time 1 s.

1. A method for the identification of a modulator of the activity of a catechol O-methyl transferase enzyme (COMT) comprising the steps of:
a) providing a COMT substrate covalently linked to a fluorescence dye,
b) contacting the molecule of step a) with a catechol O-methyl transferase enzyme (COMT), a S-adenosylmethionine (SAM) and a candidate compound and
c) measuring the fluorescence readout of the mixture of step b), wherein an altered fluorescence readout in presence of the candidate compound compared to a blank is indicative for a modulator of a catechol O-methyl transferase enzyme (COMT).

2. The method of claim 1, wherein the method is a method for the identification of a COMT inhibitor and a decreased fluorescence readout in step c) compared to a blank is indicative for a COMT inhibitor.

3. The method of claim 1, wherein the COMT substrate is 4-nitrocatechol.

4. The method of claim 1, wherein the fluorescence dye is Alexa Fluor® 488.

5. The method of claim 1, wherein the fluorescence readout in step c) is a kinetic readout.

6. The method of claim 1, wherein the COMT is human COMT.

7. The method of claim 1, wherein the method is a High Throughput screening method.

8. The method of claim 1, wherein the method is performed in a microtiter plate.

9. The method of claim 1, wherein the final concentration of COMT is about 25 nM.

10. The method of claim 1, wherein the final concentration of the COMT substrate is about 200 nM.

11. The method of claim 1, wherein the final concentration of SAM is about 500 nM.

12. A method for the identification of a substrate of a catechol O-methyl transferase enzyme (COMT) comprising the steps of:
a) providing a mixture comprising a COMT substrate covalently linked to a fluorescence dye and S-adenosylmethionine (SAM),
b) contacting the mixture of step a) with different concentrations of a candidate compound,
c) contacting the mixtures of step b) with a catechol O-methyl transferase enzyme (COMT) and
d) measuring the kinetic fluorescence readout of the mixtures of step c), wherein a decreasing fluorescence readout plateau as a function of an increasing concentration of the candidate compound is indicative for a substrate of a catechol —O-methyl transferase enzyme (COMT).

13. The method of claim 12, wherein the COMT substrate is 4-nitrocatechol.

14. The method of claim 12, wherein the fluorescence dye is Alexa Fluor® 488.

15. The method of claim 13, wherein the fluorescence dye is Alexa Fluor® 488.
